(19) United States
(12) Patent Application Publication
(10) Pub. No.: US 2011/0008372 A1
(43) Pub. Date: Jan. 13, 2011

(54) ENHANCEMENT OF LIGHT ACTIVATED DRUG THERAPY THROUGH COMBINATION WITH OTHER THERAPEUTIC AGENTS

(75) Inventor: James C. Chen, Clyde Hill, WA (US)

Correspondence Address: LAW OFFICES OF RONALD M ANDERSON, 600 108TH AVE, NE, SUITE 507, BELLEVUE, WA, 98004, US

(73) Assignee: Light Sciences Oncology, Inc., Bellevue, WA (US)

(21) Appl. No.: 12/832,494

(22) Filed: Jul. 8, 2010

Related U.S. Application Data
(60) Provisional application No. 61/223,953, filed on Jul. 8, 2009.

(51) Int. Cl. A61K 39/395; A61K 31/58; A61K 31/395; A61K 31/69; A61K 31/165; A61K 31/704; A61P 35/00

(52) U.S. Cl. 424/174.1; 514/171; 514/183; 514/64; 514/619; 514/34

(57) ABSTRACT

The efficacy of light activated therapy treatment is enhanced by use of additional therapeutic agents. Abnormal tissue is destroyed by light activated therapy, and the associated administration of one or more additional therapeutic agents can synergistically enhance the therapy. For example, the concepts disclosed herein encompass the use of the following agents in combination with light activated drug therapy: (1) agents that selectively inhibit heat shock protein 90 (Hsp90); (2) agents that inhibit the Hedgehog pathway (which is believed to play a central role in allowing the proliferation and survival of certain cancer-causing cells, and which is implicated in many of the most deadly cancers); and, (3) agents for reducing the anti-apoptotic effects of Bcl-2 or Bcl-xL. These agents can be used with light activated drug therapy individually, or in various combinations and permutations.

### RELATED APPLICATIONS

This application is based on a prior copending provisional application Ser. No. 61/223,953, filed on Jul. 8, 2009, the benefit of the filing date of which is hereby claimed under 35 U.S.C. §119(e).

### BACKGROUND

Abnormal cells in the body are known to selectively absorb certain dyes that have been perfused into a treatment site to a much greater extent than absorbed by surrounding tissue. For example, tumors of the pancreas and colon may absorb two to three times the volume of certain dyes, compared to normal cells. Once pre-sensitized by dye tagging in this manner, the cancerous or abnormal cells can be destroyed by irradiation with light of an appropriate wavelength or waveband corresponding to an absorption wavelength or waveband of the dye, with minimal damage to surrounding normal tissue. The procedure that uses light to destroy undesirable tissue, known as light activated drug therapy, has been clinically used to treat metastatic breast cancer, bladder cancer, lung carcinomas, esophageal cancer, basal cell carcinoma, malignant melanoma, ocular tumors, head and neck cancers, and other types of malignant tissue growths. Because this therapy selectively destroys abnormal cells that have absorbed more of a photoreactive dye than normal cells, it can successfully be used to kill malignant tissue with less effect on surrounding benign tissue than alternative treatment procedures.

Rather than waiting for the photoreactive agent to be selectively absorbed by the abnormal tissue, the photoreactive agent can be introduced into blood vessels in a mass of abnormal tissue, and light can be selectively directed towards such vessels to activate the photoreactive agent, thereby disrupting the blood supply in the mass of abnormal tissue, and inducing cell death by depriving the abnormal cells of oxygen and nutrients.

In typical applications, the light is administered to an internal treatment site through an optical fiber from an external source such as a laser or is applied to a site exposed during a surgical procedure. However, alternative techniques exist to provide light activated drug therapy. For example, several different embodiments of implantable light emitting probes for administering light activated therapy to an internal site within a patient's body are disclosed in commonly assigned U.S. Pat. No. 5,445,608. Further, a number of embodiments of flexible light emitting probes are disclosed in commonly assigned U.S. Pat. Nos. 5,800,478, 5,766,234, and 5,876,427. The above-referenced U.S. Pat. No. 5,445,608 teaches that an implantable probe containing a plurality of light sources can be transcutaneously introduced to a desired treatment site through a surgical incision and then left in place for an extended period of time so that the light emitted by light emitting diodes (LEDs) or other types of light sources mounted in the probe can administer light activated therapy to destroy abnormal tissue or other types of pathogenic organisms that have absorbed an appropriate photoreactive agent. Similarly, the flexible microcircuits disclosed in the above-noted patents are generally intended to be introduced into the body through a natural opening or through a small incision and positioned at the treatment site using conventional endoscopic techniques. The flexibility of these microcircuits facilitates their insertion into the body and disposition at the treatment site. Additional light emitting probes are disclosed in commonly assigned U.S. Pat. No. 6,416,531, U.S. patent application Ser. No. 11/416,783, and U.S. patent application Ser. No. 12/445,061.

It has been recognized that synergistic effects can occur when different treatment methods are combined; however, successfully predicting such synergistic effects is rare. Thus, it would be desirable to provide techniques for combining other treatment with light activated drug therapy treatment to achieve such a synergistic effect.

### SUMMARY

In accord with the concepts disclosed herein, a method is defined for more effectively destroying abnormal tissue at one or more treatment sites within a patient's body to improve the efficacy of light activated drug therapy. The method includes the step of administering a light activated drug therapy treatment to the treatment site, to destroy a portion of the abnormal tissue at the treatment site. One or more additional therapeutic agents is administered to the patient in association with the light activated drug therapy, to enhance the treatment of the abnormal tissue. In some exemplary embodiments, the additional therapeutic agent is administered to the patient after the light activated drug therapy, while in other exemplary embodiments the additional therapeutic agent is administered before the light activated drug therapy, or concurrently with the light activated drug therapy. It should be recognized that the concepts disclosed herein encompass combination and permutations of the specifically disclosed exemplary embodiments.

Significantly, light activated drug therapy delivered to a first treatment site to destroy abnormal tissue has been linked with the destruction of abnormal tissue at other treatment sites that have not been exposed to light activated drug therapy. It is believed that the abnormal tissue destroyed by light activated drug therapy at the first treatment site releases certain factors (i.e., certain biological and chemical compounds), which naturally stimulate the patient's immune system. The stimulated immune system itself is then responsible for the destruction of abnormal tissue at other treatment sites.

Administration of additional therapeutic agents can enhance the performance of light activated drug therapy in at least two ways. First, in patients with suppressed immune systems, the stimulating factor released by the abnormal tissue destroyed by light activated drug therapy at the first treatment site may be insufficient to stimulate the patient's immune system to attack abnormal tissue at other treatment sites (i.e., treatment sites not exposed to light activated drug therapy). Administration of additional therapeutic agent in association with the light activated drug therapy may provide additional stimulus to the patient's immune system, such that the patient's immune system then attacks the abnormal tissue at treatment sites that have not been treated with light activated drug therapy.

Second, even in patients whose immune system is healthy, the administration of additional therapeutic agents in association with the light activated drug therapy will provide additional stimulus to the patient's immune system, such that the patient's immune system then attacks the abnormal tissue at treatment sites that have not been treated with light activated drug therapy with greater vigor.

Many different types of additional therapeutic agents can be administered, either individually or in combination. The concepts disclosed herein encompass the use of the following agents in combination with light activated drug therapy: (1) agents that selectively inhibit heat shock protein 90 (Hsp90); (2) agents that inhibit the Hedgehog pathway (which is believed to play a central role in allowing the proliferation and survival of certain cancer-causing cells, and which is implicated in many of the most deadly cancers); and, (3) agents targeting B-cell lymphoma 2 (Bcl-2) or B-cell lymphoma-extra large (Bcl-xL) because Bcl-2 and Bcl-xL are anti-apoptotic and allows cells to evade apoptosis (by reducing the effect of Bcl-2 and Bcl-xL, the light activated drug therapy is made more effective). These agents can be used with light activated drug therapy individually, or in various combinations and permutations. Exemplary therapeutic agents are being developed by Infinity Pharmaceuticals of Cambridge, Mass.

With respect to agents that selectively inhibit Hsp90, an exemplary agent is IPI-504 (retaspimycin hydrochloride). It appears that the inhibition of Hsp90 has broad therapeutic potential for patients with solid and hematological tumors.

Yet another exemplary agent that inhibits Hsp90 is IPI-493, a propriety formulation developed by Infinity Pharmaceuticals.

With respect to agents that selectively inhibit the Hedgehog pathway, an exemplary agent is IPI-926, which is another propriety formulation developed by Infinity Pharmaceuticals. Such agents are generally analogs of cyclopamine, and they are disclosed in detail in U.S. Pat. No. 7,230,004.

With respect to agents that selectively reduce the effect of Bcl-2 and Bcl-xL, an exemplary agent ABT-737, which was developed by Abbott Laboratories, has been shown to be an antagonist of Bcl-2 and Bcl-xL. However, other agents that reduce the anti-apoptotic effect of Bcl-2 and Bcl-xL might instead be used in combination with the light activated drug therapy to achieve improved results. It has been shown that Bcl-2 and Bcl-xL are over expressed in certain types of cancer cells and tumors, which reduces the effect of apoptosis in the cells and enables the cancer cells to survive light therapy. Accordingly, administering an agent that reduces the anti-apoptotic effect of the Bcl-2 and Bcl-xL can enhance the effect of light activated drug therapy on cancer cells.

In context of the concepts discussed herein, it is hypothesized that one or more of the therapeutic agents disclosed herein, when used in conjunction with light activated drug therapy, may produce synergistic effects, enhancing the treatment of abnormal tissues.

If desired, the light activated drug therapy can be provided once, or a plurality of different times. The step of administering the light activated drug therapy treatment includes the step of administering a photoreactive agent to the treatment site. The photoreactive agent is selected for one or more characteristic wavebands of light absorption. Light having one or more emission wavebands substantially corresponding to at least a portion of the characteristic waveband of light absorption of the photoreactive agent is applied to the treatment site during each of the plurality of light activated drug therapy treatments. The light is absorbed by the photoreactive agent, which then destroys the abnormal tissue. Light can be administered from a probe implanted within the abnormal tissue, or emitted from a probe that is disposed adjacent to the abnormal tissue, using an internal or external source to produce the light.

The method may also include the step of imaging the treatment site to evaluate an effectiveness of the augmented light activated drug therapy treatment in destroying the abnormal tissue. Imaging may be accomplished using an ultrasound modality, a computer tomography modality, or a magnetic resonance imaging modality.

Another aspect of the concepts disclosed herein is using light activated drug therapy in combination with one or more of the therapeutic agents disclosed herein, such that the dosage of the therapeutic agent is relatively lower than would be employed if the therapeutic agent were to be used alone. Particularly where the relative toxicity of the therapeutic agent is high, the use of a lower dosage can result in fewer side effects, while still obtaining a beneficial outcome. Even where the relative toxicity of the therapeutic agent used in combination with the light activated drug therapy is not an issue, the cost of the therapeutic agent itself may be prohibitive, such that the use of relatively lower dosages of the therapeutic agent will result in lower health care costs. The term sub therapeutic dose is intended to refer to a dose of one of the therapeutic agents disclosed herein that is less than the dosage required to be used to obtain a therapeutic effect where the therapeutic agent is used alone. Such sub therapeutic doses will have fewer side effects. The sub therapeutic dose will be well below the maximum tolerable dose (MTD). Significantly, combination therapies generally employ the MTD, not a lower dose.

This application specifically incorporates by reference the disclosures and drawings of each patent application and issued patent identified above as a related application.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1 is a schematic illustration of a tumor, showing an implanted light source delivering a light activated therapy treatment internally to the tumor, where one or more additional therapeutic agents have been administered in association (i.e., either before, concurrently with, after, or a combination there of) with the light activated therapy; and

FIG. 2 is a schematic illustration of a tumor, showing a light source delivering a light activated therapy treatment to the tumor, where one or more additional therapeutic agents have been administered in association (i.e., either before, concurrently with, after, or a combination thereof) with the light activated therapy.

### DESCRIPTION

### Figures and Disclosed Embodiments are not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein. Further, it should be understood that any feature of one embodiment disclosed herein can be combined with one or more features of any other embodiment that is disclosed, unless otherwise indicated.

As noted above in the Background of the Invention, it is believed that damage to tumor cells resulting from administration of light activated therapy tends to attract macrophages that destroy the damaged tumor cells. Another aspect of the immunologic system relates to the response of the immunologic system to necrosis and apoptosis, for tissue destroyed by light activated therapy. Necrosis refers to the process in which cells release inflammatory agents after they have been destroyed. Apoptosis refers to cells that do not release inflammatory agents after being destroyed. The white cells or neutrophils in the body provide a scavenging function by clearing away both necrotic and apoptotic cells. It appears that abnormal tissue destroyed by light activated therapy releases factors that stimulate the patient's immune system, leading to systemic anti-tumor activity throughout a patient's body, rather than purely localized anti-tumor activity.

Thus, in a process in which light activated therapy treatment is delivered to a treatment site within a patient's body, the effectiveness of the systemic treatment can be extended by further stimulating the patient's immune system.

The method includes the step of administering a light activated drug therapy treatment to the treatment site to destroy a portion of the abnormal tissue at the treatment site. One or more additional therapeutic agents are administered to the patient in association with the light activated drug therapy. As noted above, the additional therapeutic agent (or agents, noting that such additional therapeutic agents can be used individually or in combination) can be administered before the light activated drug therapy, concurrently with the light activated drug therapy, after the light activated drug therapy, or in some combination thereof.

Administration of one or more additional therapeutic agents can enhance the performance of light activated drug therapy in at least two ways. In patients with suppressed immune systems, administration of additional therapeutic agents in association with the light activated drug therapy will provide additional stimulus to the patient's immune system, such that the patient's immune system then attacks the abnormal tissue throughout the patient's body, including at sites that have not been treated with light activated drug therapy. For such patients it may be beneficial to provide the additional therapeutic agents prior to light activated drug therapy, such that the patient's own immune system can better respond to the factors released by the abnormal tissue destroyed by light activated drug therapy, thereby enhancing the systemic effect.

In at least one embodiment, the light activated drug therapy is provided for an initial treatment of greater than about one hour. In some embodiments, the additional therapeutic agents can be provided at some time (i.e., even a period of days) after the light activated drug therapy. Subsequent analysis of the treatment effectiveness may indicate that additional light activated therapy would be desirable. Thus, the method may also include the step of imaging the treatment site to evaluate an effectiveness of the augmented light activated drug therapy treatment in destroying the abnormal tissue. Imaging may be accomplished using an ultrasound modality, a computer tomography modality, a magnetic resonance imaging modality, their combination, or any other suitable imaging modality.

In general, the step of administering the light activated drug therapy treatment includes the step of administering a photoreactive agent to the treatment site. The photoreactive agent is selected for one or more characteristic wavebands of light absorption. Light having one or more emission wavebands substantially corresponding to the characteristic waveband of light absorption of the photoreactive agent is applied to the treatment site during each light activated drug therapy treatment. The light is absorbed by the photoreactive agent, which then destroys the abnormal tissue. Light can be administered from a light source implanted within the abnormal tissue, or disposed adjacent to the abnormal tissue.

As noted above, many different types of additional therapeutic agents can be administered, either individually or in combination. The concepts disclosed herein encompass the use of the following agents in combination with light activated drug therapy: (1) agents that selectively inhibit heat shock protein 90 (Hsp90); (2) agents that inhibit the Hedgehog pathway (which is believed to play a central role in allowing the proliferation and survival of certain cancer-causing cells, and which is implicated in many of the most deadly cancers); and (3) agents for reducing the anti-apoptotic effects of Bcl-2 and Bcl-xL. These agents can be used with light activated drug therapy individually, or in various combinations and permutations. Exemplary therapeutic agents are being developed by Infinity Pharmaceuticals of Cambridge, Mass., and by others.

With respect to agents that selectively inhibit Hsp90, an exemplary agent is IPI-504 (retaspimycin hydrochloride). It appears that the inhibition of Hsp90 has broad therapeutic potential for patients with solid and hematological tumors.

Yet another exemplary agent that inhibits Hsp90 is IPI-493, a propriety formulation developed by Infinity Pharmaceuticals.

With respect to agents that selectively inhibit the Hedgehog pathway, an exemplary agent IPI-926 is another proprietary formulation developed by Infinity Pharmaceuticals. Such agents are generally analogs of cyclopamine and are disclosed in detail in U.S. Pat. No. 7,230,004.

With respect to agents that reduce the anti-apoptotic effects of Bcl-2 and Bcl-xL, an exemplary agent is ABT-737, a propriety formulation developed by Abbott Laboratories. Other examples of drugs to reduce anti-apoptotic effects include geldanamycin, PS-341 (a proteasome inhibitor bortezomib), Trichostatin A, doxorubicine, and anti-CTLA-4 antibodies, alone or in various combinations. (Note that cytotoxic T lymphocyte antigen 4 (CTLA-4) is a key negative regulator of T-cell response.)

In the context of the concepts discussed herein, applicant hypothesizes that one or more of the additional therapeutic agents disclosed herein, when used in conjunction with light activated drug therapy, may produce synergistic effects, enhancing the treatment of abnormal tissues. When used in combination with light activated drug therapy, the dose of each of the one or more additional therapeutic agents that is administered can be lowered, compared to the dose of such agents that would normally be used if the agent were administered alone and not in combination with light activated drug therapy.

In addition to enhancing systemic effects, other local (i.e., localized to the site treated with light activated therapy) advantages are believed likely to result from increasing the number of immune cells after an initial light activated therapy treatment has destroyed some of the tumor cells at a treatment site. One potential advantage is that the removal of necrotic and apoptotic tissue by the increased number of cells will likely reduce interstitial tumor pressure, thereby improving the delivery of drugs to the tumor site, particularly, the photoreactive agent employed for a successive light activated therapy treatment. In addition, the reduced interstitial tumor pressure will enhance the delivery of oxygen to the remaining tumor, by increasing blood flow to the remaining tumor. It is generally believed that singlet oxygen produced during a light activated therapy treatment is involved in the destruction of abnormal cells. The increase in oxygen delivery to the remaining tumor will thus likely increase this desired action in subsequent treatments.

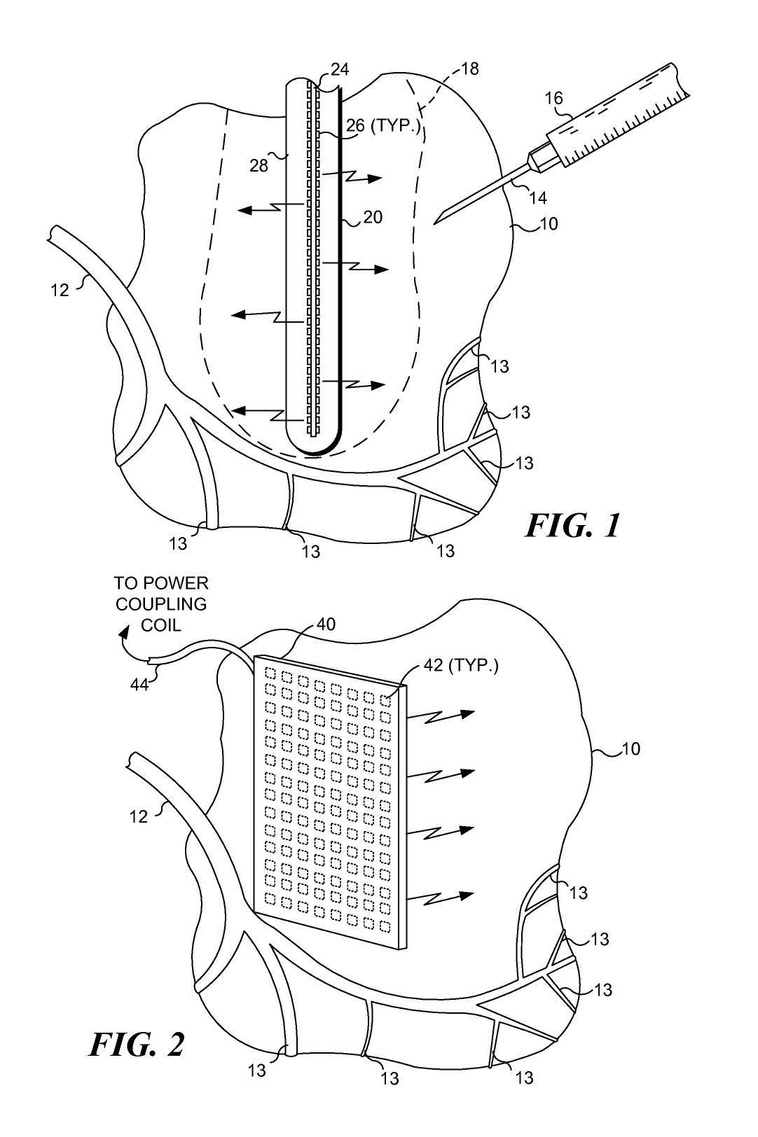

FIGS. 1 and 2 illustrate how the present invention is employed to achieve improved efficacy during the course of a plurality of light activated therapy treatments delivered to a tumor 10. In FIG. 1, tumor 10 is supplied blood through one or more main vessels 12, having a plurality of branching vessels 13. Only one such vessel is illustrated to simplify the Figure. Because the cells comprising tumor 10 are abnormal, it tends to grow at a relatively rapid rate and if left unchecked, the condition may lead to a metastatic spread of the abnormal cells throughout a patient's body.

To administer light activated therapy treatments to tumor 10 in the example shown in FIG. 1, an elongate probe 20 is implanted internally within tumor 10 during a conventional minimally invasive, surgical, or endoscopic procedure. Probe 20 may be either rigid or flexible, as appropriate to the technique used to facilitate its placement within tumor 10 and depending upon the location of the tumor within the patient's body. In this exemplary embodiment, probe 20 includes a plurality of light sources 26, e.g., LEDs, which are disposed on opposite sides of a substrate 24. Details such as the electrically conductive traces that convey electrical current to each of the light sources are not shown. An optically transparent and biocompatible sheath 28 encloses light sources 26 and substrate 24, but allows light emitted by the light sources to be transmitted through the sheath to an interior surface 18 of the tumor.

In FIG. 1, a syringe 16 is illustrated; the syringe includes a needle 14 that is inserted into tumor 10 to infuse a photoreactive agent, such as a porphyrin, into the treatment site. Alternatively, the porphyrin or other light activatable agent can be administered intravascularly. The photoreactive agent is selectively absorbed by the abnormal cells comprising tumor 10 to a much greater extent than by surrounding normal cells. Light emitted by light sources 26 has a characteristic waveband that is substantially equal to an absorption waveband of the photoreactive agent. Thus, tumor cells that have absorbed the photoreactive agent are destroyed by the light emitted from probe 20. In a related embodiment, the photoreactive agent is introduced into one or more blood vessels in the tumor, and the light from the probes activates the photoreactive agent in the blood vessels (note that such an embodiment does not require that the photoreactive agent be absorbed into the abnormal tissue).

After one or more light activated therapy treatments have been administered, syringe 16 is used to administer one or more of the above identified additional therapeutic agents (but not into the tumor as shown in FIG. 1) in multiple injections delivered over a period of time, to stimulate the patient's immune system, or enhance the treatment of the tumor in another synergistic fashion. It should be recognized that the additional therapeutic agents can be administered using techniques other than injection, and some additional therapeutic agents may require or work best using different methods of administering the agent. Thus, the specific technique employed to administer the additional therapeutic agent is not limited to injection. The stimulated immune system results in systemic destruction of abnormal cells (i.e., beyond the portion of tumor 10 treated using light activated therapy). The one or more additional therapeutic agents can alternatively be administered before, concurrently, or in a combination of before, concurrently, and/or after the light therapy is carried out.

FIG. 2 illustrates the use of a generally planar substrate probe 40 that includes a plurality of light sources 42, which again comprise LEDs in this exemplary embodiment. Light sources 42 are mounted on substrate probe 40 in a spaced-apart array that covers the surface of the substrate so that light emitted by the light sources is generally directed toward the tumor 10. A biocompatible, optically transparent sheath (not shown) encloses the light sources and the conductive traces (also not shown) that convey electrical current to the light sources to energize them. A probe that emits light conveyed through one or more optical fibers from one or more external lights sources can alternatively be used.

Syringe 16 (FIG. 1) is used for administering the photoreactive agent that is selectively absorbed by the abnormal cells comprising tumor 10 before the light is administered to the tumor from light sources 42. The light emitted by light sources 42 has a characteristic wavelength or waveband corresponding to the absorption wavelength or waveband of the photoreactive agent which is preferentially absorbed by the abnormal cells and thus kills the abnormal cells with little effect on any normal cells of the surrounding tissue. Once at least an initial light activated therapy treatment has then been delivered, killing some of the abnormal tumor cells, immune system stimulating factor/factors is administered (it being understood that the administration of other therapeutic agents to the patient can occur before, concurrently with, or after the light activated therapy).

As noted above, although not shown in either Figure, it is also contemplated that an optical fiber can be used to administer light to a treatment site (e.g., tumor 10) within the patient's body from one or more external light sources such as a laser. Other types of light sources can be used either in connection with implanted probes like those shown in FIGS. 1 and 2, or to provide light from outside the patient's body. The only significant requirement is that the light source produce light having a characteristic waveband corresponding to that of the photoreactive agent administered to the patient to implement the light activated therapy, and that such light be directed onto the abnormal tissue (or the blood vessels within the abnormal tissue) at the treatment site.

If an implanted probe is employed, electrical power can be supplied to energize the probe from outside the patient's body using an external power source that is connected to a coil applied on the outer surface of the patient's skin, generally opposite an internally implanted coil that is connected to the implanted probe (neither shown), for example, through a line 44 as illustrated in FIG. 2. A similar arrangement can be used to provide power and other signals to implanted probe 20, in FIG. 1. Other details related to the use of implanted probes and other designs for implanted probes are disclosed in the patents and patent applications identified in the Background section, above.

Synergistic effects sometimes arise when multiple therapies are combined, where the combined therapy is more effective than would have been predicted based on the effects of the individual therapies if administered alone. Such synergies are known, but are difficult to predict in advance. Applicant has hypothesized that light activated drug therapy combined with one or more of the additional therapeutic agents identified herein will produce such a synergistic effect. Further, applicant has hypothesized that light activated therapy combined with relatively low doses of the additional therapeutic agents indentified herein will produce such a synergistic effect.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A method for more effectively destroying abnormal tissue within a patient's body, comprising the steps of:
(a) administering a light activated drug therapy treatment to a treatment site within the patient's body, the light activated drug therapy treatment destroying at least a portion of the abnormal tissue at the treatment site; and
(b) administering an additional therapeutic agent to the patient in association with the light activated drug therapy treatment, said additional therapeutic agent enhancing destruction of the abnormal tissue, the additional therapeutic agent comprising one or more therapeutic agents selected from a group of additional therapeutic agents consisting of:
(i) agents that selectively inhibit a heat shock protein 90 (Hsp90);
(ii) agents that inhibit a Hedgehog pathway; and
(iii) agents for reducing anti-apoptotic effects of Bcl-2 or Bcl-xL.

2. The method of claim 1, wherein the agents for inhibiting Hsp90 comprise retaspimycin hydrochloride.

3. The method of claim 1, wherein the agents for inhibiting the Hedgehog pathway comprise an analog of cyclopamine.

4. The method of claim 1, wherein the agents for reducing the anti-apoptotic effects of Bcl-2 or Bcl-xL comprises one or more agents selected from the group consisting of geldanamycin, PS-341, Trichostatin A, doxorubicine, and anti-CTLA-4 antibodies.

5. The method of claim 1, wherein one or more therapeutic agents from the group of additional therapeutic agents is administered to the patient before administering the light activated drug therapy treatment.

6. The method of claim 1, wherein one or more therapeutic agents from the group of additional therapeutic agents is administered to the patient concurrently with administering the light activated drug therapy treatment.

7. The method of claim 1, wherein one or more therapeutic agents from the group of additional therapeutic agents is administered to the patient after administering the light activated drug therapy treatment.

8. The method of claim 1, wherein one or more therapeutic agents from the group of additional therapeutic agents is administered to the patient at multiple times, and the multiple times are selected as a combination, which may be a subset, of before, concurrently, and after administering the light activated drug therapy treatment to the patient.

9. The method of claim 1, wherein the step of administering the at least one additional therapeutic agent comprises the step of administering the at least one additional therapeutic agent at one or more times relative to a time at which the light activated drug therapy treatment is administered to achieve a synergistic improvement in destroying the abnormal tissue within the patient's body compared to using only the light activated drug therapy or only the at least one additional therapeutic agent.

10. The method of claim 1, wherein the step of administering the additional therapeutic agent comprises the step of administering the additional therapeutic agent at a dose that is lower than that normally administered if the additional therapeutic agent is not administered along with the light activated drug therapy.

11. The method of claim 1, wherein the step of administering the light activated drug therapy comprises the steps of:
(a) administering a light activatable agent having one or more characteristic light absorption wavebands to the patient, such that a quantity of the light activatable agent is present in the abnormal tissue at the treatment site; and
(b) irradiating the treatment site with light having a waveband that at least partially overlaps the one or more characteristic light absorption wavebands of the light activatable agent, activating the light activatable agent.

12. A method for more effectively destroying abnormal tissue within a patient's body, comprising the steps of:
(a) administering a light activated drug therapy treatment to the abnormal tissue at a treatment site within the patient's body, the light activated drug therapy treatment destroying at least a portion of the abnormal tissue at the treatment site; and
(b) administering one or more anti-cancer agents that cooperate synergistically with the light activated drug therapy treatment to improve an efficacy with which the abnormal tissue within the patient's body is destroyed, said anti-cancer agent thereby enhancing destruction of the abnormal tissue.

13. The method of claim 12, further comprising the step of selecting the one or more anti-cancer agents from a group of anti-cancer agents consisting of:
(a) anti-cancer agents that selectively inhibit a heat shock protein 90 (Hsp90);
(b) anti-cancer agents that inhibit a Hedgehog pathway; and
(c) anti-cancer agents for reducing an anti-apoptotic effects of Bcl-2 or Bcl-xL.

14. The method of claim 13, wherein the anti-cancer agents for inhibiting Hsp90 comprise retaspimycin hydrochloride.

15. The method of claim 13, wherein the anti-cancer agents for inhibiting the Hedgehog pathway comprise an analog of cyclopamine.

16. The method of claim 13, wherein the anti-cancer agents for reducing the anti-apoptotic effects of Bcl-2 or Bcl-xL comprises one or more anti-cancer agents selected from the group consisting of geldanamycin, PS-341, Trichostatin A, doxorubicine, and anti-CTLA-4 antibodies.

17. The method of claim 12, wherein the step of administering the one or more anti-cancer agents comprises the step of administering the one or more anti-cancer agents at one or more times selected to be one of:
(a) before the step of administering the light activated drug therapy treatment;
(b) concurrently with the step of administering the light activated drug therapy treatment;
(c) after the step of administering the light activated drug therapy treatment; and
(d) at multiple times that are a combination or a subset of time including before, concurrently, and after the step of administering the light activated drug therapy treatment.

18. The method of claim 12, wherein the step of administering the one or more anti-cancer agents comprises the step of administering the one or more anti-cancer agents at one or more times relative to the step of administering the light activated drug therapy treatment so as to maximize a synergistic effect achieved by combining administration of the light activated drug therapy with the administration of the one or more anti-cancer agents.

19. The method of claim 12, wherein the step of administering the light activated drug therapy comprises the step of administering the light activated drug therapy at the treatment site so as to cause apoptotic cell death of the abnormal tissue at the treatment site with the light activated drug therapy, while the step of administering the anti-cancer agents comprises the step of systemically administering the anti-cancer drugs to synergistically destroy the abnormal tissue throughout the patient's body.

20. The method of claim 12, wherein the step of administering the light activated drug therapy comprises the step of administering the light activated drug therapy at the treatment site at multiple times, so that removal of necrotic and apoptotic cells destroyed by an initial light activated drug therapy causes an increased number of neutrophils, which reduces interstitial tumor pressure, thereby improving a delivery of subsequently administered light activated drugs to the treatment site, for successive light activated drug therapy treatments.

21. The method of claim 12, wherein the step of administering the light activated drug therapy comprises the step of activating a light activatable drug administered to the patient, with light emitted from one or more probes that are positioned either within a mass of the abnormal tissue or adjacent thereto.

22. The method of claim 21, wherein the step of administering the light activated drug therapy comprises the step of administering the light activatable drug so that it is either absorbed by the abnormal tissue at the treatment site or is carried by blood that is circulating within vessels disposed within the abnormal tissue at the treatment site.

23. The method of claim 12, wherein the step of administering the one or more anti-cancer agents comprises the step of administering each of the one or more anti-cancer agents at a dose that is lower than that normally administered if the anti-cancer agent is not administered along with the light activated drug therapy.

24. A method for more effectively destroying abnormal tissue within a patient's body, comprising the steps of:
(a) administering a light activated drug therapy treatment to a treatment site within the patient's body, the light activated drug therapy treatment destroying at least a portion of the abnormal tissue at the treatment site; and
(b) administering an additional therapeutic agent to the patient in association with the light activated drug therapy treatment, said additional therapeutic agent enhancing destruction of the abnormal tissue, the additional therapeutic agent being administered at a reduced dosage level that is lower than a normal dosage level employed when the additional therapeutic agent is administered without the light activated drug therapy.

25. The method of claim 24, wherein the additional therapeutic agent at the reduced dosage level and the light activated drug therapy achieve a synergistic effect on the destruction of the abnormal tissue.
